SEQUENCE LISTING (15 sequences)

SEQ ID NO: 1 - protein, 451 aa, Homo sapiens
MRECISIHVGQAGVQIGNACWELYCLEHGIQPDGQMPSDKTIGGGDDSFNTFFSETGAGKHVPRAVFVDLEPTVIDEVRTGTYRQLFHPEQLITGKEDAANNYARGHYTIGKEIIDLVLDRIRKLADQCTGLQGFLVFHSFGGGTGSGFTSLLMERLSVDYGKKSKLEFSIYPAPQVSTAVVEPYNSILTTHTTLEHSDCAFMVDNEAIYDICRRNLDIERPTYTNLNRLIGQIVSSITASLRFDGALNVDLTEFQTNLVPYPRIHFPLATYAPVISAEKAYHEQLSVAEITNACFEPANQMVKCDPRHGKYMACCLLYRGDVVPKDVNAAIATIKTKRTIQFVDWCPTGFKVGINYQPPTVVPGGDLAKVQRAVCMLSNTTAIAEAWARLDHKFDLMYAKRAFVHWYVGEGMEEGEFSEAREDMAALEKDYEEVGVDSVEGEGEEEGEEY

SEQ ID NO: 2 - protein, 881 aa, Homo sapiens
MFWKFDLHTSSHLDTLLEREDLSLPELLDEEDVLQECKVVNRKLLDFLLQPPHLQAMVAWVTQEPPDSGEERLRYKYPSVACEILTSDVPQINDALGADESLLNRLYGFLQSTGSLNPLLASFFSKVMGILINRKTDQLVSFLRKKDDFVDLLLQHIGTSAIMDLLLRLLTCVERPQLRQDVVNWLNEEKIVQRLIEQIHPSKDENQHSNASQSLCDIIRLSREQMIQVQDSPEPDQLLATLEKQETIEQLLSNMFEGEQSQSVIVSGIQVLLTLLEPRRPRSESVTVNSFFSSVDGQLELLAQGALESTVSSVGALHALRPRLSCFHQLLLEPPKLEPLQMTWGMLAPPLGNTRLHVVKLLASALSANDAALTHELLALDVPNTMLDLFFHYVFNNFLHAQVEGCVSTMLSLGPPPDSSPETPIQNPVVKHLLQQCRLVERILTSWEENDRVQCAGGPRKGYMGHLTRVAGALVQNTEKGPNAEQLRQLLKELPSEQQEQWEAFVSGPLAETNKKNMVDLVNTHHLHSSSDDEDDRLKEFNFPEEAVLQQAFMDFQMQRMTSAFIDHFGFNDEEFGEQEESVNAPFDKTANITFSLNADDENPNANLLEICYKDRIQQFDDDEEEEDEEEAQGSGESDGEDGAWQGSQLARGARLGQPPGVRSGGSTDSEDEEEEDEEEEEDEEGIGCAARGGATPLSYPSPGPQPPGPSWTATFDPVPTDAPTSPRVSGEEELHTGPPAPQGPLSVPQGLPTQSLASPPARDALQLRSQDPTPPSAPQEATEGSKVTEPSAPCQALVSIGDLQATFHGIRSAPSSSDSATRDPSTSVPASGAHQPPQTTEGEKSPEPLGLPQSQSAQALTPPPIPNGSAPEGPASPGSQ

SEQ ID NO: 3 - protein, 261 aa, Homo sapiens
MNPNCARCGKIVYPTEKVNCLDKFWHKACFHCETCKMTLNMKNYKGYEKKPYCNAHYPKQSFTMVADTPENLRLKQQSELQSQVRYKEEFEKNKGKGFSVVADTPELQRIKKTQDQISNIKYHEEFEKSRMGPSGGEGMEPERRDSQDGSSYRRPLEQQQPHHIPTSAPVYQQPQQQPVAQSYGGYKEPAAPVSIQRSAPGGGGKRYRAVYDYSAADEDEVSFQDGDTIVNVQQIDDGWMYGTVERTGDTGMLPANYVEAI

SEQ ID NO: 4 - protein, 211 aa, Homo sapiens
MDNLSSEEIQQRAHQITDESLESTRRILGLAIESQDAGIKTITMLDEQKEQLNRIEEGLDQINKDMRETEKTLTELNKCCGLCVCPCNRTKNFESGKAYKTTWGDGGENSPCNVVSKQPGPVTNGQLQQPTTGAASGGYIKRITNDAREDEMEENLTQVGSILGNLKDMALNIGNEIDAQNPQIKRITDKADTNRDRIDIANARAKKLIDS

SEQ ID NO: 5 - protein, 1595 aa, Homo sapiens
MAGAWLRWGLLLWAGLLASSAHGRLRRITYVVHPGPGLAAGALPLSGPPASSRTFNVALNARYSRSSAAAGAPSRASPGVPSERTRRTSKPGGAALQGLRPPPPPPPEPARPAVPGGQLHPNPGGHPAAAPFTKQGRQVVRSKVPQETQSGGGSRLQVHQKQQLQGVNVCGGRCCHGWSKAPGSQRCTKPSCVPPCQNGGMCLRPQLCVCKPGTKGKACETIAAQDTSSPVFGGQSPGAASSWGPPEQAAKHTSSKKADTLPRVSPVAQMTLTLKPKPSVGLPQQIHSQVTPLSSQSVVIHHGQTQEYVLKPKYFPAQKGISGEQSTEGSFPLRYVQDQVAAPFQLQGVKVKFPPNIVNIHVKHPPEASVQIHQVSRIDGPTGQKTKEAQPGQSQVSYQGLPVQKTQTIHSTYSHQQVIPHVYPVAAKTQLGRCFQETIGSQCGKALPGLSKQEDCCGTVGTSWGFNKCQKCPKKPSYHGYNQMMECLPGYKRVNNTFCQDINECQLQGVCPNGECLNTMGSYRCTCKIGFGPDPTFSSCVPDPPVISEEKGPCYRLVSSGRQCMYPLSVHLTKQLCCCSVGKAGPHCEKCPLPGTAAFKEICPGGMGYTVSGVHRRRPIHHHVGKGPVFVKPKNTQPVAKSTHPPPLPAKEEPVEALTFSREHGARSAEPEVATAPPEKEIPSLDQEKTKLEPGQPQLSPGISAIHLHPQFPVVIEKTSPPVPVEVAPEASTSSASQVIAPTQVTEINECTVNPDICGAGHCINLPVRYTCICYEGYRFSEQQRKCVDIDECTQVQHLCSQGRCENTEGSFLCICPAGFMASEEGTNCIDVDECLRPDVCGEGHCVNTVGAFRCEYCDSGYRMTQRGRCEDIDECLNPSTCPDEQCVNSPGSYQCVPCTEGFRGWNGQCLDVDECLEPNVCANGDCSNLEGSYMCSCHKGYTRTPDHKHCRDIDECQQGNLCVNGQCKNTEGSFRCTCGQGYQLSAAKDQCEDIDECQHRHLCAHGQCRNTEGSFQCVCDQGYRASGLGDHCEDINECLEDKSVCQRGDCINTAGSYDCTCPDGFQLDDNKTCQDINECEHPGLCGPQGECLNTEGSFHCVCQQGFSISADGRTCEDIDECVNNTVCDSHGFCDNTAGSFRCLCYQGFQAPQDGQGCVDVNECELLSGVCGEAFCENVEGSFLCVCADENQEYSPMTGQCRSRTSTDLDVDVDQPKEEKKECYYNLNDASLCDNVLAPNVTKQECCCTSGAGWGDNCEIFPCPVLGTAEFTEMCPKGKGFVPAGESSSEAGGENYKDADECLLFGQEICKNGFCLNTRPGYECYCKQGTYYDPVKLQCFDMDECQDPSSCIDGQCVNTEGSYNCFCTHPMVLDASEKRCIRPAESNEQIEETDVYQDLCWEHLSDEYVCSRPLVGKQTTYTECCCLYGEAWGMQCALCPLKDSDDYAQLCNIPVTGRRQPYGRDALVDFSEQYTPEADPYFIQDRFLNSFEELQAEECGILNGCENGRCVRVQEGYTCDCLDGYHLDTAKMTCFDVNECDELNNRMSLCKNAKCINTDGSYKCLCLPGYVPSDKPNYCTPLNTALNLEKDSDLE

SEQ ID NO: 6 - protein, 649 aa, Homo sapiens [sequence omitted]

SEQ ID NO: 7 - protein, 536 aa, Homo sapiens
MGSNKSKPKDASQRRRSLEPAENVHGAGGGAFPASQTPSKPASADGHRGPSAAFAPAAAEPKLFGGFNSSDTVTSPQRAGPLAGGVTTFVALYDYESRTETDLSFKKGERLQIVNNTEGDWWLAHSLSTGQTGYIPSNYVAPSDSIQAEEWYFGKITRRESERLLLNAENPRGTFLVRESETTKGAYCLSVSDFDNAKGLNVKHYKIRKLDSGGFYITSRTQFNSLQQLVAYYSKHADGLCHRLTTVCPTSKPQTQGLAKDAWEIPRESLRLEVKLGQGCFGEVWMGTWNGTTRVAIKTLKPGTMSPEAFLQEAQVMKKLRHEKLVQLYAVVSEEPIYIVTEYMSKGSLLDFLKGETGKYLRLPQLVDMAAQIASGMAYVERMNYVHRDLRAANILVGENLVCKVADFGLARLIEDNEYTARQGAKFPIKWTAPEAALYGRFTIKSDVWSFGILLTELTTKGRVPYPGMVNREVLDQVERGYRMPCPPECPESLHDLMCQCWRKEPEERPTFEYLQAFLEDYFTSTEPQYQPGENL

SEQ ID NO: 8 - protein, 44 aa, Homo sapiens
MADKPDMGEIASFDKAKLKKTETQEKNTLPTKETIEQEKRSEIS

SEQ ID NO: 9 - protein, 457 aa, Homo sapiens
MTTEDYKKLAPYNIRRSSTSGDTEEEEEEEVVPFSSDEQKRRSEAASGVLRRTAPREHSYVLSAAKKSTGSPTQETQAPFIAKRVEVVEEDGPSEKSQDPPALARSTPGSNSSRGEEIVRLQILTPRAGLRLVAPDVEGMSSSATSVSAVPADRKSNSTAAQEDAKADPKGALADYEGKDVATRVGEAWQERPGAPRGGQGDPAVPAQQPADPSTPERQSSPSGSEQLVRRESCGSSVLTDFEGKDVATKVGEAWQDRPGAPRGGQGDPAVPTQQPADPSTPEQQNSPSGSEQFVRRESCTSRVRSPSSCMVTVTVTATSEQPHIYIPAPASELDSSSTTKGILFVKEYVNASEVSSGKPVSARYSNVSSIEDSFAMEKKPPCGSTPYSERTTGGICTYCNREIRDCPKITLEHLGICCHEYCFKCGICSKPMGDLLDQIFIHRDTIHCGKCYEKLF

SEQ ID NO: 10 - protein, 736 aa, Homo sapiens
MEALIPVINKLQDVFNTVGADIIQLPQIVVVGTQSSGKSSVLESLVGRDLLPRGTGIVTRRPLILQLVHVSQEDKRKTTGEENGVEAEEWGKFLHTKNKLYTDFDEIRQEIENETERISGNNKGVSPEPIHLKIFSPNVVNLTLVDLPGMTKVPVGDQPKDIELQIRELILRFISNPNSIILAVTAANTDMATSEALKISREVDPDGRRTLAVITKLDLMDAGTDAMDVLMGRVIPVKLGIIGVVNRSQLDINNKKSVTDSIRDEYAFLQKKYPSLANRNGTKYLARTLNRLLMHHIRDCLPELKTRINVLAAQYQSLLNSYGEPVDDKSATLLQLITKFATEYCNTIEGTAKYIETSELCGGARICYIFHETFGRTLESVDPLGGLNTIDILTAIRNATGPRPALFVPEVSFELLVKRQIKRLEEPSLRCVELVHEEMQRIIQHCSNYSTQELLRFPKLHDAIVEVVTCLLRKRLPVTNEMVHNLVAIELAYINTKHPDFADACGLMNNNIEEQRRNRLARELPSAVSRDKSSKVPSALAPASQEPSPAASAEADGKLIQDSRRETKNVASGGGGVGDGVQEPTTGNWRGMLKTSKAEELLAEEKSKPIPIMPASPQKGHAVNLLDVPVPVARKLSAREQRDCEVIERLIKSYFLIVRKNIQDSVPKAVMHFLVNHVKDTLQSELVGQLYKSSLLDDLLTESEDMAQRRKEAADMLKALQGASQIIAEIRETHLW

SEQ ID NO: 11 - protein, 1030 aa, Homo sapiens
MKMADAKQKRNEQLKRWIGSETDLEPPVVKRQKTKVKFDDGAVFLAACSSGDTDEVLKLLHRGADINYANVDGLTALHQACIDDNVDMVKFLVENGANINQPDNEGWIPLHAAASCGYLDIAEFLIGQGAHVGAVNSEGDTPLDIAEEEAMEELLQNEVNRQGVDIEAARKEEERIMLRDARQWLNSGHINDVRHAKSGGTALHVAAAKGYTEVLKLLIQAGYDVNIKDYDGWTPLHAAAHWGKEEACRILVDNLCDMEMVNKVGQTAFDVADEDILGYLEELQKKQNLLHSEKRDKKSPLIESTANMDNNQSQKTFKNKETLIIEPEKNASRIESLEQEKVDEEEEGKKDESSCSSEEDEEDDSESEAETDKTKPLASVTNANTSSTQAAPVAVTTPTVSSGQATPTSPIKKFPTTATKISPKEEERKDESPATWRLGLRKTGSYGALAEITASKEGQKEKDTAGVTRSASSPRLSSSLDNKEKEKDSKGTRLAYVAPTIPRRLASTSDIEEKENRDSSSLRTSSSYTRRKWEDDLKKNSSVNEGSTYHKSCSFGRRQDDLISSSVPSTTSTPTVTSAAGLQKSLLSSTSTTTKITTGSSSAGTQSSTSNRLWAEDSTEKEKDSVPTAVTIPVAPTVVNAAASTTTLTTTTAGTVSSTTEVRERRRSYLTPVRDEESESQRKARSRQARQSRRSTQGVTLTDLQEAEKTIGRSRSTRTREQENEEKEKEEKEKQDKEKQEEKKESETSREDEYKQKYSRTYDETYQRYRPVSTSSSTTPSSSLSTMSSSLYASSQLNRPNSLVGITSAYSRGITKENEREGEKREEEKEGEDKSQPKSIRERRRPREKRRSTGVSFWTQDSDENEQEQQSDTEEGSNKKETQTDSISRYETSSTSAGDRYDSLLGRSGSYSYLEERKPYSSRLEKDDSTDFKKLYEQILAENEKLKAQLHDTNMELTDLKLQLEKATQRQERFADRSLLEMEKRERRALERRISEMEEELKMLPDLKADNQRLKDENGALIRVISKLSK

SEQ ID NO: 12 - protein, 738 aa, Homo sapiens
MQPRWAQGATMWLGVLLTLLLCSSLEGQENSFTINSVDMKSLPDWTVQNGKNLTLQCFADVSTTSHVKPQHQMLFYKDDVLFYNISSMKSTESYFIPEVRIYDSGTYKCTVIVNNKEKTTAEYQLLVEGVPSPRVTLDKKEAIQGGIVRVNCSVPEEKAPIHFTIEKLELNEKMVKLKREKNSRDQNFVILEFPVEEQDRVLSFRCQARIISGIHMQTSESTKSELVTVTESFSTPKFHISPTGMIMEGAQLHIKCTIQVTHLAQEFPEIIIQKDKAIVAHNRHGNKAVYSVMAMVEHSGNYTCKVESSRISKVSSIVVNITELFSKPELESSFTHLDQGERLNLSCSIPGAPPANFTIQKEDTIVSQTQDFTKIASKSDSGTYICTAGIDKVVKKSNTVQIVVCEMLSQPRISYDAQFEVIKGQTIEVRCESISGTLPISYQLLKTSKVLENSTKNSNDPAVFKDNPTEDVEYQCVADNCHSHAKMLSEVLRVKVIAPVDEVQISILSSKVVESGEDIVLQCAVNEGSGPITYKFYREKEGKPFYQMTSNATQAFWTKQKASKEQEGEYYCTAFNRANHASSVPRSKILTVRVILAPWKKGLIAVVIIGVIIALLIIAAKCYFLRKAKAKQMPVEMSRPAVPLLNSNNEKMSDPNMEANSHYGHNDDVRNHAMKPINDNKEPLNSDVQYTEVQVSSAESHKDLGKKDTETVYSEVRKAVPDAVESRYSRTEGSLDGT

SEQ ID NO: 13 - protein, 199 aa, Homo sapiens
MANRGPAYGLSREVQQKIEKQYDADLEQILIQWITTQCRKDVGRPQPGRENFQNWLKDGTVLCELINALYPEGQAPVKKIQASTMAFKQMEQISQFLQAAERYGINTTDIFQTVDLWEGKNMACVQRTLMNLGGLAVARDDGLFSGDPNWFPKKSKENPRNFSDNQLQEGKNVIGLQMGTNRGASQAGMTGYGMPRQIL

SEQ ID NO: 14 - protein, 142 aa, Homo sapiens
MIRFILIQNRAGKTRLAKWYMQFDDDEKQKLIEEVHAVVTVRDAKHTNFVEFRNFKIIYRRYAGLYFCICVDVNDNNLAYLEAIHNFVEVLNEYFHNVCELDLVFNFYKVYTVVDEMFLAGEIRETSQTKVLKQLLMLQSLE

SEQ ID NO: 15 - protein, 1087 aa, Homo sapiens
MADHVQSLAQLENLCKQLYETTDTTTRLQAEKALVEFTNSPDCLSKCQLLLERGSSSYSQLLAATCLTKLVSRTNNPLPLEQRIDIRNYVLNYLATRPKLATFVTQALIQLYARITKLGWFDCQKDDYVFRNAITDVTRFLQDSVEYCIIGVTILSQLTNEINQADTTHPLTKHRKIASSFRDSSLFDIFTLSCNLLKQASGKNLNLNDESQHGLLMQLLKLTHNCLNFDFIGTSTDESSDDLCTVQIPTSWRSAFLDSSTLQLFFDLYHSIPPSFSPLVLSCLVQIASVRRSLFNNAERAKFLSHLVDGVKRILENPQSLSDPNNYHEFCRLLARLKSNYQLGELVKVENYPEVIRLIANFTVTSLQHWEFAPNSVHYLLSLWQRLAASVPYVKATEPHMLETYTPEVTKAYITSRLESVHIILRDGLEDPLEDTGLVQQQLDQLSTIGRCEYEKTCALLVQLFDQSAQSYQELLQSASASPMDIAVQEGRLTWLVYIIGAVIGGRVSFASTDEQDAMDGELVCRVLQLMNLTDSRLAQAGNEKLELAMLSFFEQFRKIYIGDQVQKSSKLYRRLSEVLGLNDETMVLSVFIGKIITNLKYWGRCEPITSKTLQLLNDLSIGYSSVRKLVKLSAVQFMLNNHTSEHFSFLGINNQSNLTDMRCRTTFYTALGRLLMVDLGEDEDQYEQFMLPLTAAFEAVAQMFSTNSFNEQEAKRTLVGLVRDLRGIAFAFNAKTSFMMLFEWIYPSYMPILQRAIELWYHDPACTTPVLKLMAELVHNRSQRLQFDVSSPNGILLFRETSKMITMYGNRILTLGEVPKDQVYALKLKGISICFSMLKAALSGSYVNFGVFRLYGDDALDNALQTFIKLLLSIPHSDLLDYPKLSQSYYSLLEVLTQDHMNFIASLEPHVIMYILSSISEGLTALDTMVCTGCCSCLDHIVTYLFKQLSRSTKKRTTPLNQESDRFLHIMQQHPEMIQQMLSTVLNIIIFEDCRNQWSMSRPLLGLILLNEKYFSDLRNSIVNSQPPEKQQAMHLCFENLMEGIERNLLTKNRDRFTQNLSAFRREVNDSMKNSTYGVNSNDMMS

(19) United States
(12) Patent Application Publication
(10) Pub. No.: US 2011/0124009 A1
(43) Pub. Date: May 26, 2011

(54) COMPOSITION, KIT AND METHOD FOR ASSAYING NEUROPATHY

(75) Inventors: Takeshi Iwata, Tokyo (JP); Kiyoshi Matsuno, Osaka (JP); Kazuhiro Tanahashi, Shiga (JP)

(73) Assignees: NATIONAL HOSPITAL ORGANIZATION, Tokyo (JP); Santen Pharmaceutical Co., Ltd., Osaka-shi, Osaka (JP)

(21) Appl. No.: 12/935,800

(22) PCT Filed: Mar. 31, 2009
(86) PCT No.: PCT/JP2009/056729

§ 371 Date: Jan. 3, 2011

(30) Foreign Application Priority Data
Mar. 31, 2008 (JP) 2008-091522

(51) Int. Cl. G01N 33/68; C07K 16/18; C07K 16/40; G01N 33/573; C12Q 1/48; C12Q 1/34; C12Q 1/42; B01D 59/44

(52) U.S. Cl. 435/7.4; 436/501; 435/7.9; 530/387.9; 530/387.3; 435/15; 435/18; 435/21; 250/282

(57) ABSTRACT

The present invention relates to a method for detecting a disease accompanied with neuropathy such as glaucoma, comprising measuring and/or detecting one or more of polypeptides shown in SEQ ID NOS: 1 to 15, mutants thereof, or fragments thereof in a biological sample from a subject, and also to a composition or kit for diagnosis of a disease accompanied with neuropathy such as glaucoma.

### TECHNICAL FIELD

The present invention relates to a composition or a kit useful for diagnosis of neuropathy.

The present invention also relates to a method for assaying (or determining or identifying) neuropathy using the composition or the kit.

### BACKGROUND ART

Due to progress of medical technology and changes in social environments, diseases that develop or progress in association with ageing have been highlighted in recent years. As represented by lifestyle-associated diseases, it is said that such diseases develop and progress as a result of the gradual accumulation of small changes occurring in the living body. In particular, diseases caused mainly by neuropathy have become serious social problems.

The term “neuropathy” (or a neurological disorder) refers to a condition in which stenosis and/or occlusion of peripheral blood vessels that supply oxygen and nutrients to tissues is caused by arteriosclerosis or the like, resulting in stagnation of blood flow and insufficient supply of nutrients to peripheral tissues, and eventually leading to an abnormal state of nerve functions. The development of a vascular disorder causes serious problems such as neuropathy in an organ or tissue which is rich in vasculature. If a vascular disorder develops in a sensory organ, and particularly in ocular tissue, it might result in blindness.

A fluid called “aqueous humor” flows in the eyes and serves in place of blood so as to deliver nutrition and the like. Aqueous humor is produced in the ciliary body and is discharged from the Schlemm's canal. The eye shape is maintained by the aqueous humor pressure which refers to “intraocular pressure.” Intraocular pressure slightly varies depending on season or time of day, but it is maintained at an almost constant level.

Glaucoma is a disease associated with visual field constriction and optic nerve disorder caused by a certain cause. An increase in intraocular pressure is said to be a pathological cause of the disease. The disease causes blindness of the elderly. Along with a sharp increase in the elderly population in recent years, the number of glaucoma patients has been continuously increasing.

Glaucoma is asymptomatic and thus it is difficult to detect glaucoma at an early stage. Since glaucoma can cause blindness, it is very important to diagnose the disease at an early stage. Hitherto, for the diagnosis of glaucoma, funduscopy has mainly been performed. Prior to the examination, a mydriatic agent that allows the pupil to dilate is administered to a patient, and subsequently, a physician directly observes the retina with a funduscope or fundus camera. However, when the pupils are allowed to dilate under the influence of a mydriatic agent, the flow of aqueous humor becomes stagnant, resulting in increase of intraocular pressure. Therefore, at present, it cannot be said that the use of such agent will never cause further deterioration of pathological conditions. Moreover, it cannot always be said that direct observation of the retina by a physician is an objective assay method. In direct observation, every patient must be examined by a physician, and thus it is difficult to apply such examination to mass-screening for the examination of many subjects.

As described above, there are rather many problems in relation to existing examination methods. Hence, a high-throughput assay method for early diagnosis that is less stressful for patients has been awaited, whereby the degree of pathological progression and time-dependent changes during treatment can be objectively and quantitatively determined for patients.

An assay method using a diagnosis marker is an objective high-throughput method. In the past, a method for diagnosing glaucoma using an antibody that specifically recognizes TIGR protein, which is a glucocorticoid-induced protein produced by trabecular meshwork cells, (Japanese Patent Publication (Kohyo) No. 10-509866 A (1998)), as well as quantification of TGF-β in aqueous humor (Min S H, Lee T I, Chung Y S, Kim H K., Transforming growth factor-beta levels in human aqueous humor of glaucomatous, diabetic and uveitic eyes. Korean J Ophthalmol. 2006 September; 20(3):162-5.), have been disclosed. In these methods, glaucoma cannot be determined with relatively high specificity with the use of such markers, and the ocular tissue that is not easy to take for diagnosis purpose is used as a specimen. Therefore, the methods are still research-stage methods under the present circumstances.

Along with the recent progress in genome analysis (genomics) and proteome analysis (proteomics), a variety of novel marker candidates have been reported. For glaucoma, as a result of proteome analysis using an ocular tissue, a variety of novel marker candidates have been reported (Bhuattacharya S K, Crabb J S, Bonilha V L, Gu X, Takahara H, Crabb J W., Proteomics implicates peptidyl arginine deiminase 2 and optic nerve citrullination in glaucoma pathogenesis., Invest Ophthalmol V is Sci. 2006 June; 47(6):2508-14., and Tezel G, Tang X, Cai J., Proteomic identification of oxidatively modified retinal proteins in a chronic pressure-induced rat model of glaucoma, Invest Ophthalmol V is Sci. 2005 September; 46(9):3177-3187). However, there is no report on protein markers for glaucoma found by proteome analysis using blood specimens. Also, there is no known method for diagnosing the glaucoma using protein markers in bloods from glaucoma patients. It is expected that if markers allowing diagnosis of glaucoma and diagnosis methods using such markers can be created, such markers or methods will be widely used for diagnosis of neuropathy itself.

### DISCLOSURE OF THE INVENTION

### Problem to be Solved by the Invention

However, the above-mentioned known markers and marker candidates have poor specificity and/or sensitivity, and efficient methods for detecting such markers from biological samples have not been established. Because, in general, these markers are not clinically used, there are high demands on markers with higher specificity and sensitivity for neuropathy. In addition, a high-throughput assay method that is less stressful for patients has been awaited, whereby degrees of pathological progression, as well as post-surgical time-dependent changes, can be objectively and quantitatively determined for patients.

An object of the present invention is to provide a composition or kit useful for diagnosis of a disease accompanied with neuropathy, particularly glaucoma, and a method for assaying a disease accompanied with neuropathy using the composition or kit.

### Means for Solving the Problem

The present inventors have now found blood protein markers specifically detected in glaucoma patients by subjecting blood specimens of patients with glaucoma and blood specimens of patients with another ocular diseases to proteome analysis. This finding led to the completion of an invention drawn to a method for determining glaucoma using said protein markers.

### SUMMARY OF THE INVENTION

The present invention has the following characteristics.

(1) A method for determining neuropathy, comprising quantitatively or qualitatively measuring and/or detecting one or more of polypeptides comprising any of amino acid sequences shown in SEQ ID NOS: 1 to 15, mutants thereof, or fragments thereof in a biological sample from a subject.

(2) The method according to (1), wherein the neuropathy is ocular tissue neuropathy.

(3) The method according to (2), wherein the ocular tissue neuropathy is glaucoma.

(4) The method according to any one of (1) to (3), wherein the measurement and/or detection of the polypeptide, a mutant thereof, or a fragment thereof is carried out by mass spectrometry.

(5) The method according to (4), wherein the measurement and/or detection is carried out using a substance capable of binding to the polypeptide, a mutant thereof, or a fragment thereof.

(6) The method according to (5), wherein the substance capable of binding is an antibody or an antigen-binding fragment thereof.

(7) The method according to (6), wherein the antibody labeled with any of an enzyme, a fluorophor, a dye, a radioisotope, or biotin is used.

(8) The method according to (6) or (7), wherein the antibody or an antigen-binding fragment thereof is a monoclonal antibody or a polyclonal antibody, or an antigen-binding fragment thereof.

(9) The method according to any one of (1) to (8), wherein the biological sample is blood, plasma, or serum.

(10) A composition for diagnosis and/or detection of neuropathy, which comprises one or more antibody probes selected from antibodies, antigen-binding fragments, or chemically modified derivatives thereof capable of specifically binding to at least one of polypeptides comprising any of amino acid sequences shown in SEQ ID NOS: 1 to 15, mutants thereof, or fragments thereof.

(11) A kit for diagnosis and/or detection of neuropathy, which comprises one or more antibody probes selected from antibodies, antigen-binding fragments, or chemically modified derivatives thereof capable of specifically binding to at least one of polypeptides comprising any of amino acid sequences shown in SEQ ID NOS: 1 to 15, mutants thereof, or fragments thereof.

(12) The composition or kit for diagnosis and/or detection according to (10) or (11), wherein the neuropathy is ocular tissue neuropathy.

(13) The composition or kit for diagnosis and/or detection according to (12), wherein the ocular tissue neuropathy is glaucoma.

(14) Use of one or more antibody probes selected from antibodies, antigen-binding fragments, or chemically modified derivatives thereof capable of specifically binding to at least one of polypeptide comprising any of amino acid sequences shown in SEQ ID NOS: 1 to 15, mutants thereof, or fragments thereof, in production of the kit according to any one of (11)-(13).

### DEFINITION

Terms as used herein comprise definitions as described below.

Herein, mutants of polypeptides comprising any of amino acid sequences shown in SEQ ID NOS: 1 to 15 correspond to mutants comprising a deletion(s), substitution(s), addition(s), or insertion(s) of one or more, preferably one or several, amino acids in the amino acid sequences shown in SEQ ID NOS: 1-15 or partial sequences thereof; or mutants comprising amino acid sequences showing about 80% or more, about 85% or more, preferably about 90% or more, more preferably about 95% or more, about 97% or more, about 98% or more, or about 99% or more identity with the amino acid sequences or partial sequences thereof.

The term “% identity” as used herein generally refers to the percentage (%) of the number of amino acid residues or positions that are identical in two amino acid sequences relative to the total number of amino acid residues or positions in two amino acid sequences represented when the two amino acid sequences are aligned with or without introduction of a gap. The identity between the two amino acid sequences can be determined using a mathematical algorithm. Examples of such algorithm include an algorithm described in Karlin and Altshul, Proc. Natl. Acad. Sci. USA 1990, 87: 2264 and an improved algorithm as described in Karlin and Altshul, Proc. Natl. Acad. Sci. USA 1993, 90: 5873-5877. These types of algorithms are incorporated in BLASTN, BLASTX, and the like (Altshul et al., J. Mol. Biol. 1990, 215:403). In order to obtain an amino acid sequence homologous to any one of the polypeptide amino acid sequences shown in SEQ ID NOS: 1 to 21, a BLAST protein search is carried out using the BLAST program (e.g., score=50; word length=3). In addition, gapped BLAST (Altshul et al., Nucleic Acid Res. 1997, 25: 3389) can be used to obtain a gapped alignment.

The term “several” as used herein refers to an integer of 10, 9, 8, 7, 6, 5, 4, 3, or 2.

The term “chemically modified derivative” as used herein refers to, but is not limited to, a derivative labeled with a label such as enzyme, fluorophor, dye, or radioisotope, or a derivative having chemical modification such as biotinylation, acetylation, glycosylation, phosphorylation, ubiquitination, or sulfation.

The term “composition or kit for diagnosis and/or detection” as used herein refers to a composition or kit that can be directly or indirectly used for: diagnosing and/or detecting the presence or absence of affection with a disease accompanied with neuropathy such as glaucoma, the degree of affection, the presence or absence of improvement, or the degree of improvement; or screening for a candidate substance useful for prevention, improvement, or treatment of a disease accompanied with neuropathy such as glaucoma.

The term “biological sample” used herein as a subject of detection or diagnosis refers to a sample that contains, or suspected of containing, a target polypeptide that appears along with the development of a disease accompanied with neuropathy such as glaucoma, taken from a living body (e.g., cells, tissue, or body fluid (e.g., blood, lymphatic fluid, or urine))

The term “specifically binding to” as used herein means that an antibody or an antigen-binding fragment thereof forms an antigen-antibody complex with only a target polypeptide (that is, a glaucoma marker in the present invention), a mutant thereof, or a fragment thereof, but does not substantially form such complexes with other peptidic or polypeptidic substances. As used herein, the term “substantially” means that non-specific formation of such complexes may take place, but to a minor extent.

### ADVANTAGE OF THE INVENTION

The markers for a disease accompanied with neuropathy such as glaucoma as defined in the present invention are found in a biological sample such as blood of a patient with glaucoma, but are almost not or are not found in the same of a patient with a different ocular disease such as cataract or age-related macular degeneration. The simple use of the presence or amount of such markers as an indicator provides a significant advantage that glaucoma and a disease accompanied with neuropathy can be easily detected using blood, for example.

This description includes all or part of the contents as disclosed in the description and/or drawings of Japanese Patent Application No. 2008-091522, to which the present application claims a priority.

### BEST MODES FOR CARRYING OUT THE INVENTION

The present invention will be further described specifically as follows.

<Markers for a Disease Accompanied with Neuropathy>

According to the present invention, markers for diagnosis and/or detection of a disease accompanied with (or associated with) neuropathy using the composition or kit for diagnosis or detection of a disease accompanied with neuropathy such as glaucoma are polypeptides comprising any of amino acid sequences shown in SEQ ID NOS: 1 to 15, mutants thereof, or fragments thereof.

The polypeptides comprising the amino acid sequences shown in SEQ ID NO: 1 to 15 of the present invention are listed in Table 1 below with their protein numbers (Swiss-Prot accession names and numbers.) and their properties. These polypeptides were specifically detected in plasma from patients with glaucoma, whereas they were not detected in plasmas from patients with cataract or age-related macular degeneration, or they were detected at significantly lower levels in plasmas from patients with cataract or age-related macular degeneration than in plasmas from patients with glaucoma. In addition, the amino acid sequences of these polypeptides as shown in the attached SEQUENCE LISTING are available by accessing the Swiss-Prot data bank or the like.

TABLE 1

| SEQ ID NO: | Gene name | Protein No. | Properties |
|---|---|---|---|
| 1 | TUBA1A | Q71U36 | Tubulin alpha-1A chain |
| 2 | SAPS1 | Q9UPN7 | SAPS domain family member 1 |
| 3 | LASP1 | Q14847 | LIM and SH3 domain protein 1 |
| 4 | SNAP23 | 000161 | Synaptosomal-associated protein 23 |
| 5 | LTBP1 | Q14766 | Latent-transforming growth factor |
| | | | beta-binding protein, isoform 1L |
| 6 | DBN1 | Q16643 | Drebrin |
| 7 | SRC | P12931 | Proto-oncogene tyrosine-protein |
| | | | kinase Src |
| 8 | TMSB10 | P63313 | Thymosin beta-10 |
| 9 | ZNF185 | 015231 | Zinc finger protein 185 |
| 10 | DNM1L | 000429 | Dynamin-1-like protein |
| 11 | PPP1R12A | 014974 | Protein phosphatase 1 regulatory |
| | | | subunit 12A |
| 12 | PECAM1 | P16284 | Platelet endothelial cell adhesion |
| | | | molecule |
| 13 | TAGLN2 | P37802 | Transgelin-2 |
| 14 | AP2S1 | P53680 | AP-2 complex subunit sigma-1 |
| 15 | XPO7 | Q9UIA9 | Exportin-7 |

In the present invention, all of the above target polypeptides for detection of a disease accompanied with neuropathy are characterized in that the polypeptides can be detected only in plasmas of glaucoma patients, or that the levels of the polypeptides in glaucoma patients are significantly or remarkably higher than those in cataract or age-related macular degeneration patients. As used herein, the term “significantly” refers to the presence of a statistically significant difference, wherein the significance level (p) is less than 0.05.

Therefore, when any one of, preferably two or more of, glaucoma marker polypeptide(s) is/are detected in a biological sample of a subject, the occurrence of glaucoma and neuropathy can be determined.

The polypeptides used in the present invention can be prepared by a chemical synthesis method (e.g., peptide synthesis) or a DNA recombination technique, which are conventionally used in the art. The DNA recombination techniques are preferably used in terms of the ease of procedures or purification.

First, polynucleotide sequences encoding partial sequences of the polypeptides of the present invention are chemically synthesized using an automatic DNA synthesizer. The phosphoramidite method is generally employed for such synthesis, which enables the automatic synthesis of a single-stranded DNA with a length of no more than approximately 100 nucleotides. The automatic DNA synthesizer is commercially available from, for example, Polygen or ABI.

With the use of the thus obtained polynucleotides as probes or primers, a cDNA clone of interest is obtained by known cDNA cloning; that is, by constructing a cDNA library via an RT-PCR method from poly A(+)RNA that is obtained by treating total RNA (which is extracted from a tissue of a living body, such as ocular tissue, in which the above target gene is expressed) with an oligo dT cellulose column and then performing screening of the library, such as hybridization screening, expression screening, or antibody screening. If necessary, such cDNA clone can be further amplified by the PCR method. By such procedures, cDNA corresponding to a gene of interest can be obtained.

Probes or primers are selected from sequences of 15 to 100 continuous nucleotides based on the polypeptide sequences shown in SEQ ID NOS: 1-15 and then can be synthesized as described above. Also, cDNA cloning techniques are described in Sambrook, J. and Russel, D., Molecular Cloning, A LABORATORY MANUAL, Cold Spring Harbor Laboratory Press, issued Jan. 15, 2001, Vol. 1, 7.42-7.45 and Vol. 2, 8.9-8.17 and Ausubel et al., Current Protocols in Molecular Biology, 1994, John Wiley & Sons, for example.

Next, the thus obtained cDNA clones are each incorporated into an expression vector and then prokaryotic or eukaryotic host cells transformed or transfected with the vector are cultured, so that a polypeptide of interest can be obtained from the cells or culture supernatants. In this case, a nucleotide sequence encoding a secretory signal sequence may be flanked at the 5′ end of a DNA encoding a mature polypeptide of interest, so that the mature polypeptide can be secreted extracellularly.

Vectors and expression systems are available from Novagen, Takara Shuzo, Daiichi Pure Chemicals, Qiagen, Stratagene, Promega, Roche Diagnositics, Invitrogen, Genetics Institute, and Amersham Bioscience, for example. As host cells, prokaryotic cells such as bacteria (e.g., Escherichia coli and Bacillus subtilis), yeast (e.g., Saccharomyces cerevisiae), insect cells (e.g., Sf cell), mammalian cells (e.g., COS, CHO, BHK, and NIH3T3), and the like can be used. Vectors may contain, in addition to DNA encoding the polypeptide, regulatory elements such as a promoter (e.g., lac promoter, trp promoter, P_{L} promoter, P_{R} promoter, SV40 viral promoter, 3-phosphoglycerate kinase promoter, or glycolytic enzyme promoter), an enhancer, a polyadenylation signal, a ribosomal binding site, a replication origin, a terminator, a selection marker (e.g., a drug resistance gene such as ampicillin resistance gene or tetracycline resistance gene; or a complementary auxotrophic markers such as LEU2 or URA3), and the like.

Also, to facilitate purification of a polypeptide, an expression product can also be generated in the form of a fusion polypeptide wherein a peptidic label is bound to the C-terminus or the N-terminus of the polypeptide. Examples of a typical peptidic label include, but are not limited to, a histidine repeat (His tag) comprising 6 to 10 His residues, FLAG, a myc peptide, and a GFP polypeptide.

When the polypeptides according to the present invention are produced without adding any peptidic label, examples of purification methods include ion exchange chromatography. In addition, a combination of techniques including gel filtration chromatography or hydrophobic chromatography, isoelectric point chromatography, high performance liquid chromatography (HPLC), electrophoresis, ammonium sulfate fractionation, salting-out, ultrafiltration, and dialysis may be used. Furthermore, when a peptidic label such as a histidine repeat, FLAG, myc, or GFP is bound to the polypeptide, the purification is carried out using an affinity chromatography appropriate for each peptidic label that is generally used. In this case, an expression vector that makes isolation and purification easy is preferably constructed. In particular, the expression vector is constructed such that a target polypeptide is expressed in the form of a fusion with peptidic label, and the polypeptide is prepared genetic engineeringly using the vector. By doing so, the isolation and purification of the polypeptide can be easily performed.

Purification of nucreic acids can be carried out by purification methods using agalose gel electrophoresis, DNA-binding resin column, and the like. Alternatively, because there are commercially available automated nucleic acid purification systems and nucleic acid purification kits, etc. Purification of nucleic acids may be carried out using such commercially available tools.

As defined above, mutants of the above polypeptides according to the present invention refer to mutants comprising a deletion(s), substitution(s), addition(s), or insertion(s) of one or more, preferably one or several, amino acids in the amino acid sequences shown in SEQ ID NOS: 1-15 or partial sequences thereof; or mutants comprising amino acid sequences showing about 80% or more, about 85% or more, preferably about 90% or more, more preferably about 95% or more, about 97% or more, about 98% or more, or about 99% or more identity with the amino acid sequences or partial sequences thereof. Examples of such mutants include: homologs from mammalian species different from humans; and naturally occurring mutants such as mutants based on polymorphic mutation among mammals of the same species (e.g., race), splice mutants, and natural mutants.

Also, fragments of the above polypeptides of the present invention comprise at least 7, at least 8, at least 10, or at least 15, preferably at least 20, or at least 25, more preferably at least 30, at least 40, at least 50, at least 100, at least 150, or at least 200, or all continuous amino acid residues in the amino acid sequences of the polypeptides, and retain one or more epitopes. Such fragments are capable of immunospecifically binding to antibodies or fragments thereof of the present invention. When the above polypeptides are present in blood, for example, it is assumed that the polypeptides are present as a result of cleavage and fragmentation by an enzyme existing therein such as protease or peptidase.

### <A Composition or Kit for Diagnosis or Detection of Glaucoma>

According to the present invention, the following is provided: a composition for diagnosis and/or detection of a disease accompanied with neuropathy such as glaucoma, which comprises one or more, preferably 3 or more, more preferably 5 or more, further preferably 10 or more, and most preferably 15 different antibody probes selected from among antibodies, antigen-binding fragments, or chemically modified derivatives thereof capable of specifically binding to polypeptides comprising any of amino acid sequences shown in SEQ ID NOS: 1 to 15, mutants thereof, or fragments thereof.

As used herein, the term “composition” refers not only to a simple mixture of a plurality of antibody probes but also to a combination of the same.

An antibody that recognizes a polypeptide which is a glaucoma marker is capable of specifically binding to the polypeptide via an antigen binding site of the antibody. Such antibody usable in the present invention can be prepared by conventional techniques using polypeptides having the amino acid sequences of SEQ ID NOS: 1-15, mutants thereof, or fragments thereof or using a fusion polypeptide(s) thereof as one or more immunogens. Examples of these polypeptides, mutants thereof, or fragments thereof, or fusion polypeptides include epitopes that induce antibody formation. These epitopes may be linear epitopes or epitopes with higher order structures (discontinuous epitopes). In general, an epitope capable of binding to an antibody is thought to exist on the hydrophilic surface of a polypeptide structure.

Examples of antibodies that can be used in the present invention include antibodies of any types, classes, and subclasses. Examples of such antibodies include IgG, IgE, IgM, IgD, IgA, IgY, IgG1, IgG2, IgG3, IgG4, IgA1, and IgA2.

Moreover, antibodies in all forms are induced by the polypeptides according to the present invention. When the whole or part of the polypeptide or an epitope has been isolated, both polyclonal antibody and monoclonal antibody can be prepared using conventional techniques. An example of such method is as described in Monoclonal Antibodies, Hybridomas: A New Dimension in Biological Analyses, supervised by Kennet et al., Ple num Press, New York, 1980, for example.

A polyclonal antibody can be prepared by immunizing animals such as birds (e.g., chicken) and mammals (e.g., rabbit, goat, horse, sheep, and mouse) with the polypeptide according to the present invention. The antibody of interest can be purified from the blood of immunized animals through an appropriate combination of techniques such as ammonium sulfate fractionation, ion exchange chromatography, and affinity chromatography.

A monoclonal antibody can be obtained by a technique that comprises producing a hybridoma cell line that produces a monoclonal antibody specific to each polypeptide in mice by conventional techniques. One method for producing such hybridoma cell line comprises immunizing animals with the polypeptide according to the present invention, collecting spleen cells from immunized animals, fusing the spleen cells to a myeloma cell line so as to generate hybridoma cells, and then identifying the hybridoma cell line that produces a monoclonal antibody binding to the polypeptide. The monoclonal antibody can be collected by conventional techniques.

Preparation of monoclonal and polyclonal antibodies is described in detail as follows.

### A. Preparation of Monoclonal Antibody

### (1) Immunization and Collection of Antibody-Producing Cell

An immunogen obtained as described above is administered to a mammal such as a rat, a mouse (e.g., the inbred mouse strain Balb/c), or a rabbit. The dose of the immunogen can be appropriately determined depending on, for example, the type of an animal to be immunized or the route of administration, and it is about 50 μg to 200 μg per animal. Immunization is primarily performed by injecting an immunogen subcutaneously or intraperitoneally. Also, the intervals of immunization are not particularly limited. After the primary immunization, boost immunization is carried out 2 to 10 times, and preferably 3 or 4 times, at intervals of several days to several weeks, and preferably at intervals of 1 to 4 weeks. After the primary immunization, the antibody titer of the blood serum of the immunized animal is repeatedly measured by, for example, ELISA (Enzyme-Linked Immuno Sorbent Assay). When the antibody titer reaches a plateau, the immunogen is injected intravenously or intraperitoneally to complete the final immunization. Antibody-producing cells are collected 2 to 5 days and preferably 3 days after the final immunization. Examples of antibody-producing cells include spleen cells, lymph node cells, and peripheral blood cells, and preferably spleen cells or regional lymph node cells.

### (2) Cell Fusion

Hybridoma cell lines that produce monoclonal antibodies specific to each protein can be produced and then identified by conventional techniques. A method for producing such hybridoma cell lines comprises immunizing an animal with the polypeptide of the invention, removing spleen cells from the immunized animal, fusing the spleen cells to a myeloma cell line, producing hybridoma cells therefrom, and then identifying a hybridoma cell line that produces a monoclonal antibody binding to the enzyme of interest. Myeloma cell lines to be fused to antibody-producing cells, which can be used herein, are commercially available established cell lines of animals such as mice. Preferably, cell lines to be used herein have drug selectivity so that they cannot survive in a HAT selective medium (containing hypoxanthine, aminopterin, and thymidine) in an unfused state, but they can survive only in a state fused to antibody-producing cells. Such established cell lines are preferably derived from an animal of the same species with the immunized animal. A specific example of the myeloma cell line is a P3X63-Ag.8 strain (ATCC TIB9), which is a BALB/c mouse-derived hypoxanthine•guanine•phosphoribosyl•transferase (HGPRT) deficient cell line.

Subsequently, the myeloma cell lines are fused to the antibody-producing cells. Cell fusion is carried out in a serum-free medium for animal cell culture, such as DMEM or RPMI-1640 medium, by mixing the antibody-producing cells with the myeloma cell lines at about 1:1 to 20:1 in the presence of a cell fusion accelerator. As the cell fusion accelerator, polyethylene glycol or the like having an average molecular weight ranging from 1,500 to 4,000 daltons can be used at a concentration ranging from about 10% to 80%, for example. Optionally, an auxiliary agent, such as dimethyl sulfoxide, can be used in combination to enhance the fusion efficiency. Further, the antibody-producing cells can be fused to the myeloma cell lines using a commercially available cell fusion apparatus utilizing electric stimuli (e.g., electroporation).

### (3) Selection and Cloning of Hybridoma

The hybridomas of interest are selected from the fused cells. To this end, the cell suspension is adequately diluted with, for example, a fetal bovine serum-containing RPMI-1640 medium, then the suspension is aliquoted into each well of a microtiter plate at about two million cells/well, a selection medium is added to each well, and then culture is carried out while appropriately exchanging the selection medium with the same fresh medium. The culture temperature ranges from 20° C. to 40° C. and is preferably about 37° C. When the myeloma cell is an HGPRT-deficient cell line or thymidine kinase-deficient cell line, only a hybridoma of a cell having an ability to produce an antibody and a myeloma cell line can selectively be cultured and grown in the selection medium containing hypoxanthine, aminopterin, and thymidine (i.e., the HAT medium). As a result, cells that start to grow on about day 14 after the initiation of culture in the selection medium can be obtained as hybridoma cells.

Subsequently, whether or not the culture supernatant of the grown hybridomas contains the antibody of interest is screened for. Screening of hybridomas can be carried out in accordance with conventional techniques, without particular limitation. For example, the culture supernatant in a well containing the grown hybridomas is partially sampled and then subjected to enzyme immuno assay (EIA) or ELISA or radio immuno assay (RIA). The fused cells are cloned using the limiting dilution method or the like, and monoclonal antibody-producing cells, i.e. hybridomas, are established in the end. The hybridoma is stable during culture in a basic medium, such as RPMI-1640 or DMEM, and the hybridoma can produce and secrete a monoclonal antibody that reacts specifically with a polypeptidic marker for glaucoma of the present invention.

### (4) Recovery of Antibody

Monoclonal antibodies can be recovered by conventional techniques. Specifically, a monoclonal antibody can be collected from the established hybridoma by a conventional cell culture technique, ascites development, or the like. According to the cell culture technique, hybridomas are cultured in an animal cell culture medium, such as 10% fetal bovine serum-containing RPMI-1640 medium, MEM medium, or a serum-free medium, under common culture conditions (e.g., 37° C., 5% CO₂ concentration) for 2 to 10 days, and the antibody is obtained from the culture supernatant. In the case of ascites development, about 10 millions of hybridoma cells are administered intraperitoneally to an animal of the same species as the mammal from which the myeloma cells are derived, so as to allow the hybridoma cells to grow in large quantity. After one to two weeks, the ascites or blood serum is taken from the animal.

Where antibody purification is required in the above-described method for collecting the antibody, known techniques, such as salting out with ammonium sulfate, ion-exchange chromatography, affinity chromatography, and gel filtration chromatography, may be appropriately selected or combined to obtain the purified monoclonal antibody of the present invention.

### B. Preparation of Polyclonal Antibody

When polyclonal antibodies are prepared, an animal is immunized in the same manner as described above, the antibody titer is measured on days 6 to 60 after the final immunization by enzyme immuno assay (EIA or ELISA) or radio immuno assay (RIA), and blood is taken on the day when the maximal antibody titer is measured, in order to obtain antiserum. Thereafter, the reactivity of the polyclonal antibodies in the antiserum is measured by ELISA or the like.

Also, in the present invention, an antigen-binding fragment of the above antibodies can also be used. Examples of antigen-binding fragments that can be produced by conventional techniques include, but are not limited to, Fab and F(ab′)₂, Fv, scFv, and dsFv. Examples thereof also include antibody fragments and derivatives thereof that can be produced by genetic engineering techniques. Examples of such antibodies include synthetic antibodies, recombinant antibodies, multi-specific antibodies (including bispecific antibodies), and single chain antibodies.

The antibodies of the present invention can be used in vitro and in vivo. In the present invention, the antibodies can be used in assays for detection of the presence of polypeptides or (poly)peptide fragments thereof. A monoclonal antibody is preferably used to enable specific detection in the assay. Even in the case of a polyclonal antibody, a specific antibody can be obtained by a so-called absorption method that comprises binding an antibody to an affinity column to which a purified polypeptide is bound.

Therefore, the composition of the present invention can contain at least one, preferably a plural number of types of (e.g., two or three types or more), and more preferably all types of antibodies or antigen-binding fragments thereof capable of specifically binding to the polypeptides comprising amino acid sequences of SEQ ID NOS: 1-15, mutants thereof, or fragments thereof.

A label, such as a fluorophore, an enzyme, or a radioisotope may be bound to an antibody or an antigen-binding fragment thereof to be used in the present invention, if necessary.

Examples of a fluorophore include fluorescein and a derivative thereof, rhodamine and a derivative thereof, dansyl chloride and a derivative thereof, and umbelliferone.

Examples of an enzyme include horseradish peroxidase and alkaline phosphatase.

Examples of a radioisotope include iodines (¹³¹I, ¹²⁵I, ¹²³I, and ¹²¹I), phosphorus (³²P), sulfur (³⁵S), and metals (e.g., ⁶⁸Ga, ⁶⁷Ga, ⁶⁸Ge, ⁵⁴Mn, ⁹⁹Mo, ⁹⁹Tc, and ¹³³Xe).

Examples of other labels include luminescence substances such as luminol and bioluminescence substances such as luciferase and luciferin.

Also, if necessary, an avidin-biotin system or a streptavidin-biotin system can also be used herein. In this case, for example, biotin can be bound to the antibody or an antigen-binding fragment thereof of the present invention.

The present invention further provides a kit for diagnosis and/or detection of neuropathy, preferably ocular neuropathy, more preferably glaucoma, which comprises one or more antibody probes selected from antibodies or antigen-binding fragments thereof, or chemically modified derivatives thereof capable of specifically binding to at least one polypeptide comprising any of the amino acid sequences shown in SEQ ID NOS: 1 to 15, a mutant thereof, or a fragment thereof.

In this context, the present invention further provides a use of one or more antibody probes selected from among antibodies or antigen-binding fragments thereof, or chemically modified derivatives thereof capable of specifically binding to at least one polypeptide comprising any of the amino acid sequences shown in SEQ ID NOS: 1 to 15, a mutant thereof, or a fragment thereof for production of the above-described kit.

The kit comprises, for examples, individual containers (e.g., vials) in which the above-described antibody probes for detection of glaucoma markers are packaged individually or, appropriately, in admixture. Preferably, antibody probes may be packaged in the lyophilized state in containers.

Alternatively, the kit of the present invention may comprise a solid-phase support comprising a multi-well plate, an array, a microtiter plate, a test piece, spherical carriers such as latex beads or magnetic beads, or the like, to which antibodies or fragments thereof capable of specifically binding to the aforementioned polypeptides have been attached or (covalently or non-covalently) bonded.

Further, the kit of the present invention may contain a buffer, a secondary antibody, instructions, and the like, which are used in the assay method of the present invention.

Instead of the above-described antibody probes, nucleic acid probes can be used in the method, composition, and kit of the present invention. The nucleic acid probes are DNAs, which code for polypeptides comprising any of amino acid sequences shown in SEQ ID NOS: 1-15 as described above, mutants thereof, or fragments thereof. The nucleic acid probes can be produced by the above-described method for producing the corresponding polypeptides by gene recombination technology. Mutants or fragments thereof can be produced by, for example, PCR using appropriate primers and parent polypeptides as templates. The nucleic acid probes can generally have a size of approximately 15-100 nucleotides or more and preferably approximately 20-80 nucleotides. In addition, with the use of nucleic acid probes, DNA-DNA hybridization, DNA-RNA hybridization, RNA-RNA hybridization, or the like is performed under stringent conditions such that target markers are detected. Regarding hybridization conditions, for example, conditions described in Sambrook, J. and Russel, D., Molecular Cloning, A LABORATORY MANUAL, Cold Spring Harbor Laboratory Press, published on Jan. 15, 2001, Vol. 1, 7.42-7.45, Vol. 2, 8.9-8.17, or Ausubel et al., Current Protocols in Molecular Biology, 1994, John Wiley & Sons, etc. can be employed.

<Detection of a Disease Accompanied with Neuropathy>

According to the present invention, a disease accompanied with neuropathy can be detected by a method that comprises determining in vitro the presence or amount of one or more of the polypeptides comprising the amino acid sequences shown in SEQ ID NOS: 1-15, mutants thereof, or fragments thereof in a biological sample from a subject using substances capable of binding to the above-described markers. In a possible diagnosis conducted by the method of the present invention, where the glaucoma marker(s) is/are detected or the gene expression levels are determined to be significantly higher than control levels, a subject is determined to be in the advanced stage of neuropathy, thus to suffer from ocular neuropathy, particularly glaucoma.

In the method of the present invention, the detection of markers for a disease accompanied with neuropathy may be performed using a single marker, but is preferably performed using a plurality of (e.g., from 2 or more, 3 or more, 4 or more, or 5 or more, to 22) markers. This is intended to avoid unpredictable detection of a non-specific complex, in other words, misdiagnosis.

The composition or kit of the present invention is useful for diagnosis, determination, or detection of a disease accompanied with neuropathy, i.e., for diagnosis of the presence or absence of the disease or the degree of the disease. In diagnosis of a disease accompanied with neuropathy, comparison is made with negative controls such as normal cells, normal tissues, or normal body fluids, and then the presence or amount of the above-described glaucoma markers in a biological sample from a subject is detected. When a difference in the presence or amount is found to be significant, the subject is suspected of advanced neuropathy or suffering from glaucoma.

Examples of test samples used in the present invention include body fluids such as blood, serum, blood plasma, and urine.

Examples of the above-described substances capable of binding to glaucoma markers include not only the above-described antibodies or antigen-binding fragments thereof but also, for example, aptamers, Affibody™ (Affibody), receptors of the glaucoma markers, substances inhibiting the specific action of the glaucoma markers, and substances activating the specific action of the glaucoma markers, preferably antibodies or antigen-binding fragments thereof or chemically modified derivatives thereof.

In an embodiment of the present invention, the measurement can comprise the steps of: bringing an antibody or fragment thereof, which may be optionally labeled with a conventional enzyme or fluorophore, into contact with a tissue section or a homogenized tissue or a body fluid; and qualitatively or quantitatively measuring an antigen-antibody complex. The detection is carried out by, for example, a method for measuring the presence and the level of a target polypeptide by immunoelectron microscopy, or a method for measuring the presence or the levels of target polypeptides by a conventional method such as an enzyme antibody method (e.g., ELISA), a fluorescent antibody technique, a radioimmunoassay, a homogeneous method, a heterogeneous method, a solid phase method, or a sandwich method. Where the target polypeptide is found to be present in a body fluid or an glaucoma tissue or cells, preferably blood, obtained from a subject, or the level of the target polypeptide is found to be significantly increased or higher than the negative control level, the subject is determined to have glaucoma. As used herein, the term “significantly” refers to the presence of a statistically significant difference (p<0.05).

An example of a measurement method as an alternative for an immunological method is a method using mass spectrometry. This method can be performed specifically by procedures described in the Examples. Specifically, a biological sample such as serum or blood plasma is filtered using a filter to remove contaminants, diluted with a buffer (e.g., pH, about 8), and then adjusted to have a concentration ranging from about 10 mg/ml to about 15 mg/ml. Subsequently, the resultant is filtered through a hollow fiber filter (Reference Example (1) below) or a centrifugal flat membrane filter, which is capable of removing proteins with a molecular weight of 50,000 or more, so as to perform molecular weight fractionation. The fractions are treated with protease (e.g., trypsin) for peptidization and then the resultants are subjected to a mass spectrometer (the type using matrix-assisted laser desorption ionization or electrospray ionization). Differences between the amount of a polypeptide existing in a sample of a patient with glaucoma and the same of a healthy subject or a patient with a different ocular disease can be measured based on the mass-to-charge ratio (m/z) and intensity at a specific peak from the polypeptide of interest.

### EXAMPLES

The present invention will be described in more detail with reference to the examples set forth below; however, the technical scope of the present invention is not limited to the examples.

### Reference Example

### (1) Preparation of Hollow Fiber Filter

A hundred polysulfone hollow fibers having a pore size (molecular weight cut off) of approximately 50,000 on the membrane surface were packed into a bundle. The both ends of the bundle were fixed to a glass tube using an epoxy-based potting agent so as not to occlude the hollow parts of the hollow fibers, so that a mini module is prepared. The mini module (module A) was used for removal of high-molecular-weight proteins in serum or blood plasma, having a diameter of about 7 mm and a length of about 17 cm. Similarly, a mini module (module B) to be used for concentrating low-molecular-weight proteins was prepared using a membrane with a pore size (molecular weight cut off) of approximately 3,000. The mini modules have an inlet that is connected to hollow fiber lumen on one end and an outlet on the other end. The inlets and outlets of hollow fibers constitute flow passages of a closed circulatory system formed via a silicon tube. Through the flow passages, a liquid is driven by a Peristar pump to circulate. Also, a glass tube of the hollow fiber mantle is provided with a port for discharging a liquid leaking from the hollow fibers, so that one module set is constituted. The modules were connected to a position in the middle of such flow passage via a “T”-shaped connector, i.e., three modules A and one module B were connected in tandem, thereby forming one hollow fiber filter. The hollow fiber filter was washed with distilled water, and then filled with an aqueous 25 mM ammonium bicarbonate solution (pH 8.2). A fraction raw material (i.e., serum or blood plasma) was injected from the flow passage inlet of the hollow fiber filter and then discharged from the passage outlet after fractionation and concentration. Serum or blood plasma injected to the hollow fiber filter was applied to a molecular sieve with a molecular weight cut off of approximately 50,000 for every module A. Thus, components with molecular weights lower than that of 50,000 are concentrated using the module B and then prepared.

### Example 1

### (1) Identification of Plasma Proteins in Normal Tension-Glaucoma Patients, Cataract Patients, and Age-Related Macular Degeneration Patients

Heparinized plasmas were obtained for measurement, from 10 patients with age-related macular degeneration (aged 82 on average), 10 patients with cataract and 10 patients with normal tension-glaucoma of similar age. The blood plasmas were centrifuged to remove contaminants, and the resulting plasmas were further diluted with 25 mM ammonium bicarbonate solution (pH 8.2) to a concentration of 12.5 mg/ml, followed by carrying out a molecular weight fractionation using the hollow fiber filter as described in Reference Example (1). Each fractionated blood plasma sample (total amount of 1.8 ml, comprising 250 μg (max) of proteins) was separated into 3 fractions by reversed-phase chromatography with AKTA explorer 10s (GE Healthcare Biosciences). The fractions were each lyophilized and then redissolved in 8 M urea solution. The samples were treated with DTT•iodoacetamide and then diluted 10-fold, followed by overnight digestion at 37° C. with trypsin (at a ratio 1:50 of trypsin to proteins) for peptidization. After removal of urea using a desalting column, peptides in each fraction were further fractionated into 8 fractions using an ion-exchange column. Each resulting fraction was further fractionated using a reverse-phase column, and the eluted peptides were subjected to mass spectrometry with an online-connected mass spectrometer (LCQ Deca XP plus; Thermo Fisher Scientific K.K.).

### (2) Comparison of the Expressed Plasma Proteins Among Normal Tension-Glaucoma Patients, Cataract Patients, and Age-Related Macular Degeneration Patients

Data determined in (1) above were analyzed using the protein identification softwares Bioworks (Thermo Fisher Scientific K.K.) and Phenyx (GENE BIO) for comprehensive protein identification. From among identified proteins, proteins identified by the two different types of software were listed and designated as proteins detected from plasma samples of patients with each disease. This was carried out to exclude false-positive proteins contained in analysis results of either one of the softwares by combining the two different softwares having different algorithms. However, unlike Bioworks, Phenyx carries out searching in consideration of isoform-specific amino acid sequences obtained by alternative splicing of an identical protein and changes in mass after post-translation modification. Hence, the software Phenyx might identify peptides that cannot be identified by Bioworks. Under the above conditions, proteins identified only by Phenyx were also listed.

Among proteins listed for each disease, proteins detected from normal tension-glaucoma patients but never from cataract patients or age-related macular degeneration patients were found as plasma marker proteins. These proteins correspond to polypeptides comprising any of amino acid sequences shown in SEQ ID NOS: 1 to 15 in Table 1 (above) and the SEQUENCE LISTING. Accordingly, it was revealed that the proteins are useful as glaucoma markers for detection of glaucoma or for diagnostic determination of the progression of glaucoma during treatment.

### INDUSTRIAL APPLICABILITY

The present invention provides the compositions or kits with good specificity and sensitivity for diagnosis of a disease accompanied with neuropathy such as glaucoma, and it is particularly useful in the pharmaceutical and medical industries.

All publications, patents, and patent applications cited herein are incorporated herein by reference in their entirety.

1. A method for determining neuropathy, comprising quantitatively or qualitatively measuring and/or detecting at least one polypeptide comprising an amino acid sequence shown in SEQ ID NOS: 1 to 15, a mutant thereof, or a fragment thereof, in a biological sample from a subject.

2. The method according to claim 1, wherein the neuropathy is ocular tissue neuropathy.

3. The method according to claim 2, wherein the ocular tissue neuropathy is glaucoma.

4. The method according to claim 3, wherein the measurement and/or detection of the polypeptide, mutant thereof, or fragment thereof is carried out by mass spectrometry.

5. The method according to claim 4, wherein the measurement and/or detection is carried out with a substance capable of binding to the polypeptide, mutant thereof, or fragment thereof.

6. The method according to claim 5, wherein the substance capable of binding is an antibody or an antigen-binding fragment thereof.

7. The method according to claim 6, wherein the antibody is present and is labeled with any of an enzyme, a fluorophor, a dye, a radioisotope, or biotin.

8. The method according to claim 6, wherein the antibody or an antigen-binding fragment thereof is a monoclonal antibody or a polyclonal antibody, or an antigen-binding fragment thereof.

9. The method according to claim 1, wherein the biological sample is blood, plasma, or serum.

10. A composition for diagnosis and/or detection of neuropathy, which comprises at least one antibody probe selected from the group consisting of antibodies, antigen-binding fragments, and chemically modified derivatives thereof capable of specifically binding to at least one polypeptide comprising any amino acid sequence shown in SEQ ID NOS: 1 to 15, mutant thereof, or fragment thereof.

11. A kit for diagnosis and/or detection of neuropathy, which comprises at least one antibody probe selected from the group consisting of antibodies, antigen-binding fragments, and chemically modified derivatives thereof capable of specifically binding to at least one polypeptide comprising any amino acid sequence shown in SEQ ID NOS: 1 to 15, mutant thereof, or fragment thereof.

12. The composition according to claim 10, wherein the neuropathy is ocular tissue neuropathy.

13. The composition according to claim 12, wherein the ocular tissue neuropathy is glaucoma.

14. A method of producing the kit according to claim 11, comprising adding to a container at least one antibody probe selected from the group consisting of antibodies, antigen-binding fragments, and chemically modified derivatives thereof capable of specifically binding to at least one polypeptide comprising any amino acid sequence shown in SEQ ID NOS: 1 to 15, mutant thereof, or fragment thereof.

15. The kit according to claim 11, wherein the neuropathy is ocular tissue neuropathy.

16. The kit according to claim 15, wherein the ocular tissue neuropathy is glaucoma.
